(19) United States
(12) Patent Application Publication
(10) Pub. No.: US 2004/0254494 A1
(43) Pub. Date: Dec. 16, 2004

(54) Method and appartaus for use in nerve conduction studies

(76) Inventor: Eleonora S. Spokoyny, Newport Coast, CA (US)

Correspondence Address: Howard E. Sandler, Suite 100, 23793 Rockfield Blvd., Lake Forest, CA, 92630, US

(21) Appl. No.: 10/460,583

(22) Filed: Jun. 11, 2003

(51) Int. Cl. A61B 5/05

(52) U.S. Cl. 600/547

(57) ABSTRACT

A method and apparatus for improving the accuracy of nerve conduction studies, while simultaneously decreasing the time and skill required to undertake such studies. This improvement is accomplished by an “inching” template of the present invention which facilitates the exact location of electrodes for a series of EMG readings which must be taken along the nerve being studied to determine whether or not impingement of such nerve exists and, if so, where such impingement occurs.

### FIELD OF THE INVENTION

This invention relates to an improvement in the manner of performing nerve conduction studies to diagnose entrapment neuropathies and, more particularly, to a method and apparatus for improving the accuracy of nerve conduction studies, while simultaneously decreasing the time and skill required to undertake such studies.

### BACKGROUND OF THE INVENTION

The present invention relates to a method and apparatus for use in performing sensory and motor nerve conduction studies.

It has long been known that the electrical conduction velocity of a nerve depends on the anatomic and physiologic integrity of a nerve. Certain diseases and/or injuries affecting peripheral nerves are accompanied by a decrease in nerve conduction velocity across the involved segment of the nerve.

Sensory and motor nerve conduction studies are often used to diagnose such conditions as carpal tunnel syndrome, cubital tunnel syndrome, tarsal tunnel syndrome, compression of the peroneal nerve across the fibular head and other compression neuropathies. Diminished conduction velocity across a particular segment of the tested nerve is indicative of the site of entrapment or compression. Such studies may be used to diagnose or to follow up on an abnormal condition. Such studies could, therefore, be used to permit corrective action to be undertaken before permanent damage to the nerve occurs.

In one example, nerve conduction studies are used to diagnose suspected carpal tunnel syndrome. Carpal tunnel syndrome is usually a painful condition frequently associated with repetitive use of the hands and wrists. It may also be associated with systemic medical conditions such as Diabetes Mellitus, Rheumatoid Arthritis, thyroid dysfunction, and others. Carpal tunnel syndrome is caused by compression of the median nerve as it passes through the carpal tunnel. Carpal tunnel syndrome is characterized by pain and paresthesia in the sensory distribution of the median nerve in the hand. Symptoms include numbness, tingling and a painful burning sensation in the fingers which can radiate up the forearm, to the shoulder, as well as weakness and atrophy of the muscles innervated by the median nerve on the hand.

The invention herein is for the purpose of making diagnosis quicker and more accurate, not only for carpal tunnel syndrome, but also in instances of cubital tunnel syndrome (ulnar compressive neuropathy), posterior tarsal tunnel syndrome, to diagnose entrapment of the lateral and/or median plantar nerves, anterior tarsal tunnel syndromes (compression of the peroneal nerve at the ankle), compression of the peroneal nerve across the fibular head, and other conditions.

Nerve conduction velocity measurements are made by stimulating the peripheral nerve with an electrical impulse and measuring the time or latency from the stimulation until an action potential recorded. Measurements are made by the use of surface electrodes positioned over the muscle that picks up the signals which are then amplified and displayed in a suitable manner, such as on a computer monitor, screen of a cathode ray tube or an oscilloscope. The time between the electrically applied stimulus and recorded action potential, is known as latency. Conduction velocity is measured as distance from the cathode of the electrical stimulator to the active recording electrode, divided by latency. This technique is well known in the art and is referred to as nerve conduction velocity study (NCS) (See U.S. Pat. No. 4,291,705 entitled NEUROMUSCULAR BLOCK MONITOR, which issued on Sep. 29, 1981 to Severinghaus et al, and U.S. Pat. No. 4,807,643 entitled DIGITAL ELECTRONEUROMETER, which issued on Feb. 28, 1989 to Rosier; disclosing methods and apparatus for performing conduction studies, the disclosure of which are hereby incorporated by reference.) Reference is also made to U.S. Pat. No. 5,327,592 to Lemmon which closely describes an operational environment similar to the instant invention.

Nerve Conduction Studies (NCS) generally require sophisticated and expensive equipment. The studies are typically performed by highly trained medical personnel. Proper procedures and precise technique must be followed or error will be present in the results. For example, in conducting a median sensory nerve conduction study, the distance between the stimulating cathode and the recording electrode is critical to obtaining results, which are then compared with generally accepted standards or norms, based on earlier measurements of the normal population with reference to their gender, age, focal temperature and height. This procedure is highly enhanced by determining nerve conduction velocities at several locations along the longitudinal course of the nerve being investigated, and this is often accomplished by a technique commonly referred to as “inching” or “centimetering”. Heretofore the rather basic necessity of accurately placing stimulating electrodes and insuring the measurements of the distance of such consecutively placed electrodes along the course of the tested nerve segment are accurate and consistent, has been the source for errors, as well as very time consuming for the physician or technician completing the testing. Accordingly, a need exists for an apparatus and method which reduces the potential for measurement errors and assists in the proper placement of electrodes for nerve conduction studies using the “inching” technique. In addition, a need exists for an apparatus and method which permits electrophysiologists to conduct studies on a regular basis, at reduced cost, with greater accuracy, and at a significantly faster pace.

### SUMMARY OF THE INVENTION

The present invention includes a template, carried on a flexible body member, for use in diagnosing focal peripheral neuropathies when using the “inching technique” to obtain a spaced array of conduction velocity readings for precise localization of nerve entrapment. The template includes locating means thereon which are used to efficiently and accurately determine electrode placement for nerve velocity measurements along the nerve being investigated.

By means of the present invention, the heretofore problems of prior inching techniques are greatly alleviated. More specifically, the template of the present invention provides a fast and very accurate way of locating consecutive points of stimulation along the longitudinal extent of the nerve, being investigated so that areas of nerve compression, if any, can be readily determined.

Accordingly, it is one object and advantage of the present invention to provide an inexpensive and easy method of completing a nerve conduction examination.

It is another object and advantage of the present invention to increase the efficacy and accuracy of the operator conducting an NCS examination.

It is still another object and advantage of the present invention to provide individuals performing nerve conduction tests with an inexpensive and accurate tool to greatly reduce such operators time in performing such tests, while simultaneously increasing the accuracy and consistency of such tests.

These and other objects and advantages of the present invention will become more readily apparent upon a review of the following description and drawings, in which:

### BRIEF DESCRIPTION OF THE DRAWINGS

FIG. 1 is a plan view of one embodiment of an inching template constructed in accordance with the principles of the present invention and which is used in performing nerve conduction studies;

FIG. 2 is an edge view of the template of FIG. 1, illustrating one embodiment of a layered construction of the inching template seen in FIG. 1;

FIG. 3 is an elevational view of a surface type anode and cathode assembly of a type used in lo conjunction with the inching template of the present invention;

FIG. 4 is an plan view of a lower forearm, wrist and hand of a subject showing an inching template of the present invention in position to cooperate with the anode and cathode assembly of FIG. 3; and

FIG. 5 is a graph of a patient diagnosed with carpal tunnel syndrome, which was produced using the inching template of FIGS. 1 & 2 and positioned on the patient in a manner illustrated in FIG. 4.

### DETAILED DESCRIPTION OF A PREFERRED EMBODIMENT

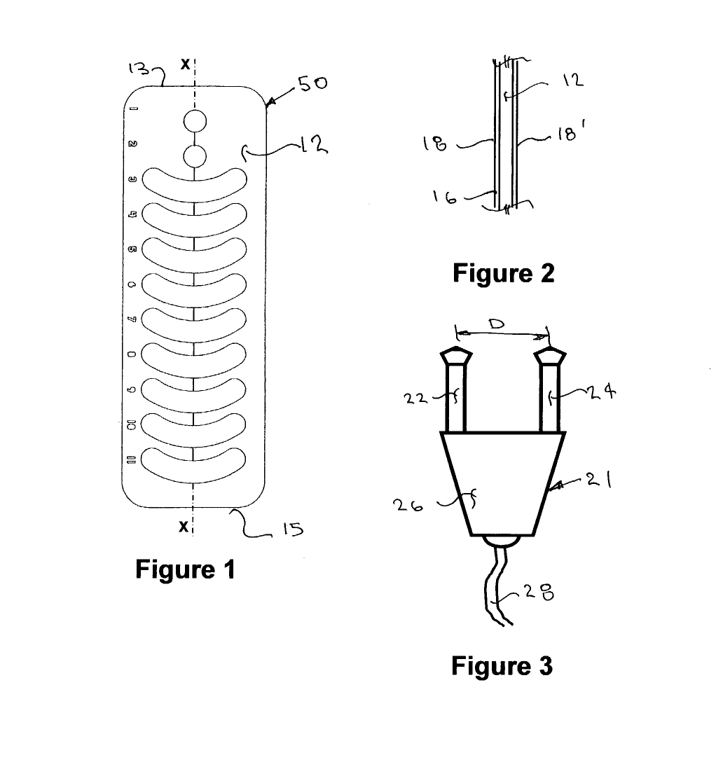

Referring now to FIG. 1 there is illustrated one embodiment of an inching template 50 of the present invention which comprises a body portion 12, which extends along longitudinal axis X-X, includes upper and lower ends 13 and 15, respectively, and is formed of a thin flexible elastomeric material. The elastomeric material forming body portion 12 may be of any suitable substance; however, it will preferably possess the following attributes: readily flexible along all axes; hypo-allergenic; non-conductive; inexpensive; thin; resistant to stretching when exposed to the normal conditions of nerve conduction studies; and moisture resistant. It is to be noted, if preferred, a suitable paper type material for body portion 12 may be an acceptable substitute for the elastomeric material discussed immediately above. In such a latter event, it would be preferred that the paper material still retain the attributes discussed above with respect to an elastomeric material. It can also be formed of a relatively thicker flexible material, than the thin material currently illustrated in FIG. 1.

In practice the template 50 will be releasably affixed to the patient in the vicinity of the nerve being studied. In this regard, FIG. 2 illustrates one preferred embodiment of body portion 12 as having a hypo-allergenic, non-aggressive adhesive coating 16 on the lower side thereof, which is the side thereof which will be adjacent to the skin of the patient when the template is releasably affixed thereto. As shown, body portion 12 additionally includes inner and outer protective films 18 and 18′, respectively, carried thereon which act to maintain the cleanliness of the body portion 12, as well as to protect the adhesive coating 16. Protective films 18 and 18′ are removed before use of the template 50 with a patient. At this point, it is to be noted that there is no necessity that an adhesive coating 16 be adhered to the entire, nor even that a portion of such surface have an adhesive coating.

In this regard, the elastomeric material of body portion 12 may have a sufficient coefficient of friction, between inner surface thereof and the skin that it is in contact with, to prevent, or at least alleviate, the tendency to slide during the nerve conductive study. As a still further alternative, to an adhesive coating on all or a portion of such inner surface of body portion 12, would be to provide a non-integrated means, such as non aggressive hypo-allergenic tape or an adjustable velcro sleeve, to releasably secure the template in position on the skin of the patient.

FIG. 3 illustrates an electrode assembly 21 which comprises: stimulating electrodes shown as anode and cathode electrodes 22 and 24, respectively; and a probe carrier 26 which carries electrodes 22 and 24, in a fixed transversely spaced relationship, at a distance of “D” apart from one another.

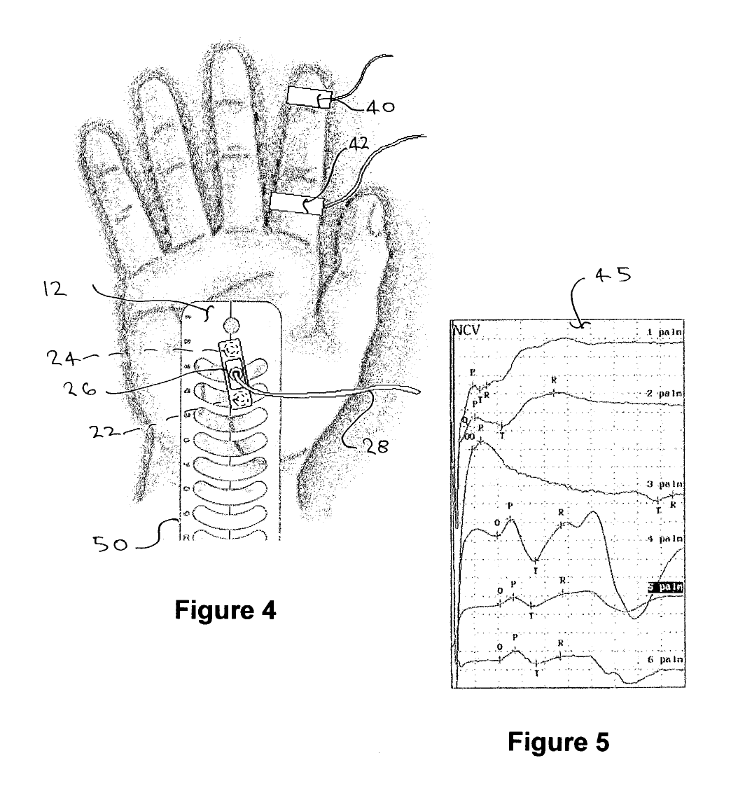

FIG. 4 illustrates the lower forearm, wrist and hand of a subject undergoing an antidromic sensory nerve conduction study of the median nerve for possible carpal tunnel syndrome and shows an inching template 50 in position and extending generally longitudinally along the wrist. Template 50 includes electrode locating openings 1-11 therethrough. As will be discussed hereinafter, locating openings are cooperable with electrodes 22 and 24 to obtain the sensory nerve action potentials, by using the inching technique.

Stimulating electrodes 22 and 24 are shown as being carried by probe carrier 26 and are fixed with respect to each other at a constant distance designated as “D”. The signals obtained by electrodes 40 and 42 are carried by cables to EMG equipment (not shown), for processing and for the generation of an appropriate display, such as graph 30 illustrated in FIG. 5, which is indicative of the pathological process of the medial nerve within the carpal tunnel. At this point it is to be noted that, inasmuch as the invention herein relates to the inching template 50, as well as the use thereof in nerve conduction studies and, further, the operation and construction of an EMG and the electrode assembly 21, and the analysis of the readings obtained therefrom, are expected to be well known to one skilled in the art of electromyography and electrodiagnostic medicine, detailed description thereof is not necessary for a full and complete understanding of this invention by such skilled persons.

Referring once again to FIG. 1, locating means, shown as comprising circular openings 1 and 2 and nine arcuate extending openings 3-11, all of which are positioned symmetrically along the axis X-X and extend transversely therethrough, serve to locate and position the electrodes 22 and 24, when performing a test using the template 50. As shown, opening 1, is downwardly adjacent the upper end 13, opening 2 is longitudinally downwardly spaced therefrom along axis X-X, arcuate opening 3 is longitudinally downwardly spaced from opening 2, and so on. The probe spacing D is closely related to the openings 1-11, as follows: the longitudinal spacing between the openings 1 & 2 is D/2; the longitudinal spacing between openings 2 & 3, as well as between the adjacent openings 4-11 is D/2, and the radius of the arc of openings 3-11 is D. It is to be noted that the diameter of the openings 1 & 2, as well as the width of the arcuate openings 3-11 are large enough to readily permit contact of the electrodes 22 and 24 with the skin. As is normal, a conductive gel is applied intermediate the ends of electrodes 22 and 24 and the adjacent skin areas to provide better electrical conductivity therebetween.

When applying an inching template 50, in a manner as illustrated in FIG. 4, in testing for carpal tunnel syndrome, while performing antidromic sensory nerve conduction study, the starting point is located, in a usual manner, and the instance illustrated the opening 1 is positioned at such starting point. The cathode 24 is positioned in opening 1 and a stimulating electrical pulse to the patient's skin, is imparted via anode 22. Cathode 24 is pivoted within the opening 1, while simultaneously having anode 22 move through the arcuate opening 3 while also maintaining contact with the skin of the patient being evaluated. The ability of having such arcuate movement allows the anode 22 to better follow and locate position over the nerve being tested and to reduce stimulus artifact. This arrangement allows the recording of a distally applied electrical signal, which has been propagated down the nerve in response to the stimulus pulse from the anode 22 (see normally applied and located well known stationary ring type reference and recording electrodes 40 and 42). The elapsed time between the applied electrical stimulus and an initial deflection from the horizontal line of the recorded action potential is expressed in milliseconds and, since the template 50 assures the absolute accuracy of the distance between various testing points, the calculation of the nerve conduction velocity is accurately obtained by dividing the measured distance by the measured elapsed time (latency).

To complete the carpal tunnel syndrome study using the template 50 and the “inching” technique, the cathode 24 will be moved to the next opening 2-9, as the case may be and be pivoted so that the anode 22 is moved at a radius D through the next arcuate opening 4-11, respectively. This procedure continues seriatim along the nerve proximally, with the cathode 24 being located centrally along axis X-X, to obtain readings at every distance with increment of D/2 (in the example shown) in a continuing expanding process. This method is used to determine the location of the focal compression of the tested nerve. All readings generated by the EMG machine are then graphed in a manner that the examiner can determine focal delay of the action potential, along the course of the tested nerve and to document nerve's impingement site, if any. To even further facilitate the placement of cathode 24, an indicia of the axis X-X is printed, laser marked or burned, or formed along the central longitudinal extent of template 50.

FIG. 5 illustrates a typical graph 45 which may be generated using the instant invention in conjunction with an EMG machine. Graph 45 illustrate the latency in the horizontal axis, against the amplitude of the sensory nerve action potential on the vertical axis. In this regard, and as can be seen at the graph, stimulations were performed along the median nerve across the carpal tunnel. Recording and referential ring electrodes 40 and 42 were positioned over the right index finger, for example 3 cm apart. Mid palm stimulation was recorded as “1 palm”. The second stimulation site was determined by the next longitudinal opening located at 1.3 cm proximally from the first one. Sensory nerve action potentials from each stimulation site were recorded on a screen with simultaneous automatic measurement of the peak and onset latencies, amplitudes and conduction velocities calculated for each stimulation. In the provided example significant delay of the action potential occurred between the third and fourth stimulating sites which corresponds to the segment located approximately 8.6 cm and 9.9 cm proximally of active recording electrode or approximately 1.0-1.5 cm distally to distal wrist crease. Latency from expected linear prolongation of 0.2-0.3 ms suddenly demonstrated delay of more than 2.0 ms. This corresponds with significant drop in conduction velocity from approximately 50-55 m/s to 6.1 m/s. This result clearly localizes abnormality to the portion of the carpal tunnel distal to the wrist crease. Equipped with such detailed localization hand surgeon would be able to perform less invasive but significantly more accurate operation for correction of the abnormality.

In view of the above description, those of ordinary skill in the art may envision various modifications, often of the sort comparable with the many alternatives discussed hereinabove by applicant at various portions of the description. As such, it is to be noted that such modifications will not depart from the inventive concepts disclosed herein and, further, the above description should not be considered as the only embodiment, or preferred embodiment of the invention herein. The true spirit and scope of the present invention may only be determined within the scope of the appended claims, in which:

1. A neurological testing template for assistance in diagnosing focal peripheral neuropathy disorders when using the “inching technique” to position an anode and cathode assembly, to obtain a spaced array of conduction velocity readings for use in the localization of nerve entrapment, comprising:
an elongated flexible, generally non-conductive, body member adapted to be in constant relationship to an extremity, having nerves being examined, in a manner that the axis of elongation of said body member extends generally in the direction of elongation of such extremity and of the selected nerve being examined;
said body member having a series of longitudinally spaced transversely extending openings therethrough which are cooperative with such anode and cathode assembly such that, during an inching procedure, the cathode and anode of such assembly are in conductive contact with the skin of such extremity through a longitudinally spaced pair of said openings and are selectively moved longitudinally along said body member and positioned to be in conductive contact with such skin through other pairs of said openings to obtain conductive readings along such selected nerve being examined.

2. A neurological testing template as specified in claim 1 wherein such constant relationship is by means of releasably securing said body member to such an extremity.

3. A neurological testing template as specified in claim 2 wherein said body member has a generally non-irritating mildly adhesive coating on at least a portion of the side thereof intended to be adjacent the skin of such extremity and said coating provides such releasable securing of said testing template to adjacent skin.

4. A neurological testing template as specified in claim 2 wherein said side thereof has said coating on substantially the entire surface thereof.

5. A neurological testing template as specified in claim 1 wherein the anode and cathode of such assembly are in fixed relationship with respect to each other, and the spacing between longitudinally spaced adjacent pairs of said openings are a whole number fraction of such fixed relationship.

6. A neurological testing template as is specified in claim 5 wherein such whole number fraction is in the range of one fourth to one half.

7. A neurological testing template as specified in claim 6 wherein said whole number fraction is one half.

8. A neurological testing template as is specified in claim 5 wherein at least some of such openings are transversely extending arcuate openings, with the arcuate ends thereof being equidistant from said longitudinal axis of said body member.

9. A neurological testing template as is specified in claim 8 wherein the radius of the arcuate centerline of said arcuate openings is equal to the center to center distance between said anode and cathode.

10. A neurological testing template as is specified in claim 8 wherein said body member contains indicia thereon indicative of the accumulated spacing between said openings.

11. A neurological testing template as is specified in claim 1 wherein said body member is formed of a generally non-conductive elastomeric material.

12. A neurological testing template as is specified in claim 1 wherein said body member is formed of a generally non-conductive paper like material.

13. In a method for neurological testing in diagnosing focal peripheral neuropathy disorders when using the inching technique by moving an anode and cathode assembly to obtain a series of readings along the nerve being examined, for conveyance to an EMG machine to determine localization of nerve entrapment, such anode and cathode being spaced a fixed distance D from one another, the improvement comprising:
providing an elongated flexible template with a plurality of equally longitudinally spaced openings extending transversely therethrough;
releasably positioning such a template on a patient in a manner to extend generally longitudinally along the nerve being examined;
positioning such anode and cathode assembly within a first pairs of such openings which are spaced “D” apart;
repositioning such anode and cathode assembly within other pairs of such openings which are spaced “D” apart;
obtaining electrical indications at each of such positioning and repositioning indicative of conduction velocity of the nerve being examined.
